(19) United States
(12) Patent Application Publication
(10) Pub. No.: US 2004/0053817 A1
(43) Pub. Date: Mar. 18, 2004

(54) Delayed-release pharmaceutical formulations

(76) Inventors: Detlef Mohr, Biberach (DE); Tim Seiffert, Solingen (DE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/416,173

(22) PCT Filed: Nov. 8, 2001
(86) PCT No.: PCT/GB01/04980

(30) Foreign Application Priority Data
Nov. 8, 2000 (DE) 100558577

(51) Int. Cl. A61K 38/28; A61K 9/14

(52) U.S. Cl. 514/3; 424/486

(57) ABSTRACT

The invention relates to a pharmaceutical delayed-release formulation containing proinsulin C-peptide and the use of the delayed-release formulations for treating of diabetes. In particular, the invention relates to delayed-release formulations in which proinsulin C-peptide is present in an absorbable matrix consisting for example of absorbable polymers. The invention also relates to microparticles which contain proinsulin C-peptide.

The invention relates to delayed-release pharmaceutical formulations containing proinsulin C-peptide.

Human proinsulin C-peptide which is formed when insulin is synthesised from proinsulin was regarded as biologically inactive for many years. However, very recently, various studies have shown that human proinsulin C-peptide may be pharmacologically effective in the treatment of diabetes and complications of diabetes such as peripheral neuropathy and nephthropathy (Johansson, Diabetic Medicine 17 (2000) 1; Wahren, J. Int. Med. 240 (1996) 115) and in the treatment of CNS disorders and for influencing the QT interval in the heart.

Because of the short half-life of plasma in humans, which is only about 40 minutes, C-peptide has to be administered continuously to the patient by subcutaneous long-term infusion (Forst, Exp Clin Diabetes 106 (1998) 270). However, this is hardly a possibility for diabetes sufferers who require constant C-peptide substitution.

C-peptide fragments or variants as described in International Application WO 98/133384 also have only a short biological half-life.

There was therefore a need for pharmaceutical formulations which contain proinsulin C-peptide and release the active substance continuously over a longer period.

The problem underlying the invention was thus to provide a pharmaceutical formulation of C-peptide which is suitable for treating chronic diseases.

The problem was solved according to the invention by preparing a delayed-release pharmaceutical formulation which contains proinsulin C-peptide and which is exceptionally suitable for the long-term therapy of chronic diseases.

The terms “human proinsulin C-peptide” or “human C-peptide” in this patent application refer to a peptide with the sequence EADLQVGQVELGGGPGAGSLQPLALEGSLQ and pharmaceutically acceptable salts of the peptide.

The terms “proinsulin C-peptide” or “C-peptide” in this patent application refer to human proinsulin C-peptide or a biologically active analogue, derivative or fragment or pharmaceutically acceptable salt thereof.

The term “biologically active fragment of human proinsulin C-peptide” denotes a peptide fragment according to patent application WO 98/13384. In particular, the term relates to the peptide fragments EGSLQ, GSLQ, ELGGPGA, ELGG, ELGGP and GGPGA and peptides containing these peptide fragments.

The term “biologically active analogue of human C-peptide” denotes a peptide produced by conservative amino acid substitution from human C-peptide, while retaining the biological activity of human C-peptide. The biological activity of these C-peptide analogues can easily be measured by determining the Na⁺—K⁺-ATPase activity. A process for this determination is described in WO 98/13384. Another possible way of determining the biological activity of C-peptide is to measure the endothelial nitric oxide synthase (eNOS)-stimulating activity of C-peptide (Kunt et al, Exp. Clin Endocrinol Diabetes 106 (1998) 270).

The term “conservative amino exchange” denotes that one or more amino acids of human C-peptide are replaced by amino acids with a similar side chain. Families of amino acids with a similar side chain include, for example, amino acids with

basic side chains (e.g. lysine, arginine, histidine)

acid side chains (e.g. aspartic acid, glutamic acid)

uncharged polar side chains (e.g. glycine, asparagine, glutamine, serine, threonine, tyrosine, cysteine)

nonpolar side chains (e.g. alanine, valine, leucine, isoleucine, proline, phenylalanine, methionine, trytophan)

branched side chains (e.g. threonine, valine, isoleucine) and

aromatic side chains (e.g. tyrosine, phenylalanine, tryptophan, histidine).

Preferred analogues of human proinsulin-C-peptide are those wherein amino acids with small side chains are exchanged for one another, e.g. glycine for serine.

By the term “biologically active derivative of human C-peptide” is meant, in this patent application, a peptide which is obtained from human proinsulin C-peptide by the modification of a side chain while retaining the biological activity. Examples of this include modifications of the N-terminal amino group (e.g. by acetylation or by the addition of polyethyleneglycols), the carboxy-terminal group (e.g. by reduction, the introduction of an amino group or esterification) or one or more amino acid side groups (e.g. by hydroxylation, phosphorylation, acetylation, deamidation, reduction, oxidation, amination, halogenation, alkylation).

The term “pharmaceutically acceptable salts” denotes salts which retain the biological activity of C-peptide and do not produce any undesirable toxic effects. Examples of such salts are addition salts of inorganic or organic acids such as e.g. hydrochloric, phosphoric acid, acetic acid, tartaric acid, fumaric acid, malic acid, succinic acid or citric acid, salts of the anions thereof and salts of C-peptide with metal cations.

As discussed above in relation to the analogues of human C-peptide, convenient methods exist for determining the biological activity of these derivatives and salts. In all cases it will be appreciated that the analogues derivatives and salts may not have the same activity as C-peptide itself while still retaining a useful biological activity. For example an increase in half life may more than compensate for a modest reduction in absolute activity. The analogues, derivatives and salts will preferably have at least 50%, typically at least 70% of the biological activity of human proinsulin C-peptide.

The term “delayed-release pharmaceutical formulation” in this patent application denotes a pharmaceutical preparation from which the active substance is released in a therapeutically relevant amount under physiological conditions over a period of at least 24 hours. Preferred delayed-release formulations are those from which proinsulin C-peptide is released in therapeutically relevant amounts over a period of at least 5 days, preferably at least 10 days and most preferably at least 14 days. The word “delayed” does not imply that there must be an initial period of no release.

A therapeutically relevant “release” is conveniently measured in vivo by measuring the circulating plasma concentration of C-peptide. Any increase over the patient's basal circulating C-peptide plasma concentration (as measured prior to commencement of treatment) may be therapeutically relevant. However, preferably a “therapeutically relevant” C-peptide plasma concentration will exceed 0.5 ng/ml (0.15 nM/ml), preferably it will exceed 0.7 ng/ml, e.g. about 1 ng/ml (0.3 nM/ml). In typical pharmacokinetic profiles, as shown by the Figures hereto, an initial burst in release of C-peptide is followed by a prolonged phase of sustained release.

In a preferred embodiment the delayed-release pharmaceutical formulation contains proinsulin C-peptide itself.

In another particularly preferred embodiment, the delayed-release pharmaceutical formulation contains an acetate salt of C-peptide. Pyroglutamate C-peptide is a further preferred active agent for inclusion in a delayed-release formulation.

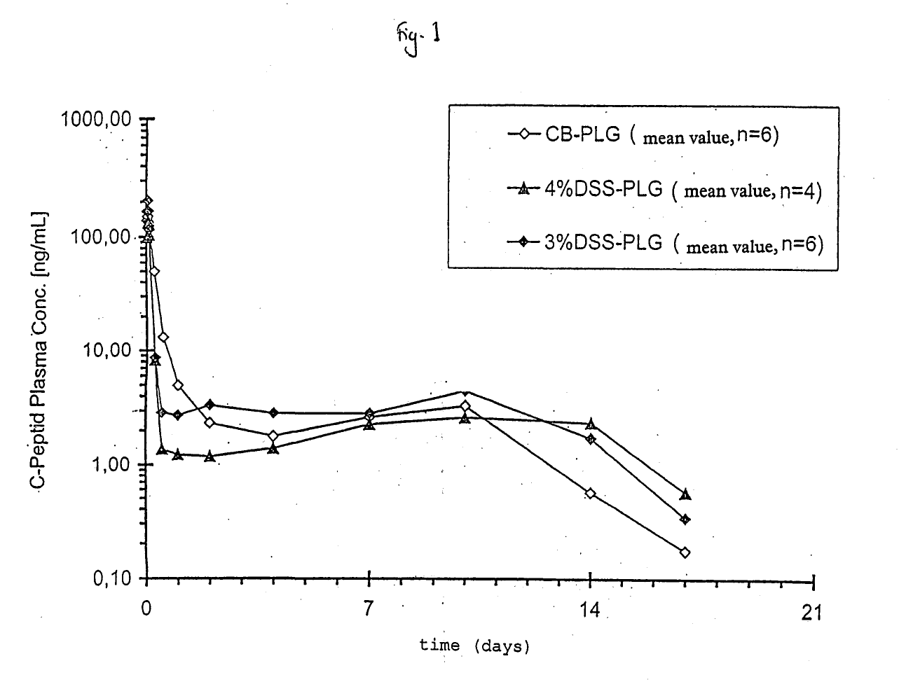

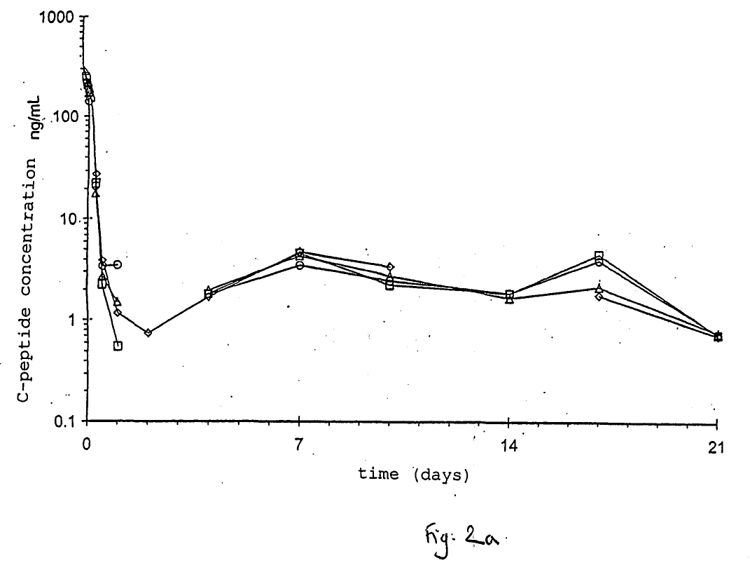

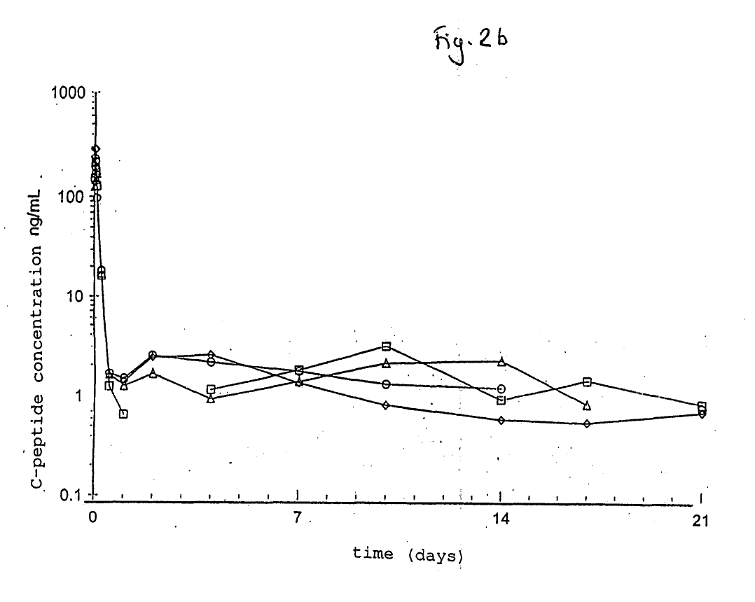

In order to delay the preferably parenterally administered C-peptide, the C-peptide was embedded in an absorbable matrix which significantly slows down the release of the peptide after administration to the patient and makes it possible to control the release of the C-peptide (cf. FIGS. 1-2).

The invention therefore relates to a delayed-release pharmaceutical formulation containing proinsulin C-peptide, characterised in that proinsulin C-peptide is present in an absorbable matrix.

Preferably, the absorbable matrix consists of absorbable polymers, or mixtures of absorbable polymers, in which the C-peptide is embedded in dispersed or dissolved form.

Polymers suitable for the preparation of delayed-release pharmaceutical formulations are known to those skilled in the art and include, for example, polylactide, polyglycolide, polylactide glycolide, polyanhydrides, polyorthoesters, polyacetals, polylactic acid, polyglycolic acid, polylactic acid polyglycolic acid, polycarbonates, polyether esters, cellulose derivatives, cyclodextrines, polyacrylates, polycaprolactones, polyvalerolactone, polypropiolactone, polybutyrolactone, polypivalolactone or polyester amides, of which polymers containing glycolide and/or lactide units, e.g. polylactide glycolide copolymers (PLG polymers), are preferred.

Particularly preferred matrix polymers are PLG polymers, especially those with a lactide/glycolide ratio of 75:25 to 25:75. D-,L- and/or D,L-lactide may be used.

Surprisingly, it has been found that the release of C-peptide from the delayed-release pharmaceutical formulations shows a particularly advantageous release profile if PLG polymers of two or preferably three different molecular weights are combined with one another.

In a preferred embodiment of the invention, the delayed-release pharmaceutical formulation therefore contains proinsulin C-peptide and an absorbable matrix, the absorbable matrix being made up of a mixture of at least two, preferably at least three different PLG polymers of different molecular weights.

Preferably, the polymer matrix contains

(A) 50-80% of a first polymer A with an average molecular weight of between 25 and 35 kDa

(B) 10-30% of a second polymer B with an average molecular weight of between 12 and 20 kDa (e.g. between 15 and 20 kDa)

(C) 10-20% of a third polymer C with an average molecular weight of between 1.5 and 3 kDa.

Preferably the formulations comprise 60-80% of a polymer of type (A), the remainder being made up of a mixture of one or more polymers of type (B) and (C). Polymers of type (A) and (B) conveniently have a lactide/glycolide molar ratio of about 50:50.

Most particularly preferred is a mixture of PLG polymers the polymer constituents of which are standardised for preparation in delayed-release pharmaceutical formulations. Examples include:

Polymer A: RG 503 or RG 503H (Boehringer Ingelheim)

Polymer B: RG 502 or RG=-502H (Boehringer Ingelheim)

Polymer C: Mn 2300 (Boehringer Ingelheim).

In another aspect of the invention, the absorbable matrix of the delayed-release formulation according to the invention contains polymers prepared by ring-opening polymerisation of lactide and/or glycolide units. These two species are preferably mixed at a ratio of 75:25 to 25:75, preferably 60:40 to 40:60.

Surprisingly, it was found that the polymerisation of lactide and/or glycolide in an extruder, especially in the presence of large amounts of catalyst of more than 1000 ppm of tin (II) and/or in the presence of electrolyte-containing polyols as moderators, e.g. a suitably charge balanced dextran sulphate such as dextran sodium sulphate (DSS), produces PLG polymers which have particularly advantageous release profiles for C-peptide.

The invention therefore provides a delayed-release pharmaceutical formulation containing C-peptide and an absorbable polymer matrix, the polymer matrix being comprising a PLG polymer produced by ring-opening polymerisation in an extruder. Preferably the polymer matrix consists essentially of one or more PLG polymers.

In particularly preferred embodiments polymerisation was carried out in the extruder using at least 1000 ppm of tin(II) based on the amount of lactide/glycolide put in.

In another preferred embodiment, polymerisation was carried out in the extruder in the presence of a moderator, most preferably in the presence of dextran sodium sulphate (DSS). Preferably 1 to 5 wt. % (e.g. 2 to 4%) of DSS is added to the prepolymer mix.

Alternatively viewed, the present invention provides a delayed-release pharmaceutical formulation containing proinsulin C-peptide and an absorbable polymer matrix obtainable by mixing lactide and glycolide units under conditions which allow polymerisation thereof. Preferably the units are mixed in an extruder and ring-opening polymerisation takes place. Preferably polymerisation takes place in the presence of at least 1000 ppm of a tin (II) catalyst (e.g. tin(II)2-ethylhexanoate) and/or an electrolyte-containing polyol e.g. DSS) which acts as a moderator.

By “moderator” in this patent application is meant a substance which is added before the polymerisation process when preparing an absorbable polyester and which influences the molecular weight, the breakdown rate and the release properties of corresponding formulations without acting as an independent polymerisation catalyst and without being incorporated in the polyester or bound thereto in any substantial amount. Typically less than 1% of the polymer matrix is made up of the moderator, preferably <0.5%, e.g. 0.001 to 0.2% of the polymer matrix is moderator.

With the PLG polymers it has been possible to prepare an injectable delayed-release form of proinsulin C-peptide which allows the proinsulin C-peptide to be released in therapeutically effective (relevant) amounts over a period of several days. At the same time, it is released surprisingly uniformly, which is ideally suited to the delayed-release administration of proinsulin C-peptide. A burst in the release of proinsulin C-peptide may be observed for the first day or two after administration, thereafter release (as reflected by circulating plasma levels) is surprisingly uniform.

The invention therefore relates to the use of proinsulin C-peptide, particularly human proinsulin C-peptide, in the manufacture of a delayed-release pharmaceutical formulation. The proinsulin C-peptide is released in therapeutically relevant amounts from a delayed-release pharmaceutical formulation of this kind according to the invention over at least 1 day, 5 days, 10 days or, preferably, at least 14 or 21 days, for example.

The delayed-release pharmaceutical formulations containing proinsulin C-peptide according to the invention may be in the form of implants, films, patches, pellets, granules, microcapsules or microparticles.

Delayed-release pharmaceutical formulations which contain microparticles containing proinsulin C-peptide and an absorbable matrix are preferred.

The preparation of delayed-release preparations of this kind is known per se in the art.

However, when preparing formulations containing human proinsulin C-peptide, it was surprisingly found that owing to the strong water-solubility of human C-peptide, methods of preparation based on phase separations lead to unacceptably high losses of active substance. Examples of less suitable processes of this kind connected with phase separations are multiple emulsion processes, e.g. W/O/W or W/I/O processes.

Therefore, methods of preparing the delayed-release preparations in which the charging of the polymers with active substance is carried out in single-phase systems or wherein the solvent phase is evaporated off are preferred. Examples of suitable methods include the preparation of implants, films or pellets by C-peptide charging of the polymers in extruders or the preparation of particles of active substance by ASES methods as described in European Patent EP 563 176.

Particularly preferably, the delayed-release forms according to the invention are prepared by processing polymer/proinsulin C-peptide mixtures by spray drying to form microparticles. For this, the polymer mixture is dissolved in a solvent, the polypeptide is also dissolved and stirred into the polymer solution. Finally, the solution is sprayed until the polymer/polypeptide mixture is precipitated in particle form.

The invention therefore relates to microparticles consisting of an absorbable polymer matrix in which proinsulin C-peptide, most preferably human proinsulin C-peptide, is dispersed, dissolved or suspended. Microparticles measuring not more than 10 μm are preferred.

The invention also relates to delayed-release pharmaceutical formulations comprising microparticles which contain proinsulin C-peptide and an absorbable matrix, the microparticles being prepared by spray drying.

The delayed-release formulations according to the invention may contain a proportion of proinsulin C-peptide of 1-50% (w/w) based on the dry content, while a proinsulin C-peptide content of 1-30% (w/w) and most particularly 3-15% (w/w) is preferred.

The proportion of the absorbable matrix in the delayed-release formulation according to the invention is between 5 and 99% (w/w) of the dry mass, preferably between 50 and 97% (w/w) and most preferably between 80 and 96% (w/w).

In a typical example the dry formulation consists of

90% of absorbable polymers and

10% of human proinsulin C-peptide.

This dry formulation can be taken up before use in a pharmaceutically acceptable solvent which also contains other ingredients which are well known to those skilled in the art of parenteral drug preparations. Examples are buffers, electrolytes, stabilisers, preservatives and/or detergents.

Alternatively, the pharmaceutical excipients may already be present in the dry formulation, which may then have the following composition, for example:

80% polymer

7% proinsulin C-peptide

3.25% NaCl

0.5% detergent

5.75% cellulose derivative

0.5% preservative

3% buffer

A formulation of this kind would preferably be reconstituted before use by taking it up in electrolyte-free solvent, e.g. in pyrogen-free water.

The pharmaceutical formulation ready for use then contains, for example:

277.5 mg of polymer

22.5 mg of human proinsulin C-peptide

13 mg of NaCl

1.5 mg of Tween

12 mg of carboxymethylcellulose

1.5 ml of water

The polymer and the proinsulin C-peptide preferably being in the form of microparticles and the proinsulin C-peptide preferably being dissolved or dispersed in the polymer matrix.

The invention also relates to the use of the pharmaceutical formulations of proinsulin C-peptide according to the invention for treating diabetes and complications of diabetes, particularly diabetic neuropathy, diabetic nephthropathy, diabetic retinopathy or to reduce the electrocardiographic QT interval.

The delayed-release formulations according to the invention are intended particularly for parenteral administration. Preferred methods of administration are by subcutaneous, intramuscular and intracutaneous route.

Subcutaneous administration is particularly preferred.

The pharmaceutical formulation according to the invention may contain, in addition to proinsulin C-peptide, another pharmacologically active substance which is suitable for the prevention, treatment or alleviation of diabetes or complications of diabetes.

Useful examples of these are combinations with insulin, insulin derivatives or insulin-like growth factor, antidiabetics, painkillers, neurotrophines such as Nerve Growth Factor, or ACE inhibitors. These additional active substances may be present in the same formulations or in separate formulations, e.g. as a kit-of-parts, for simultaneous, separate or sequential administration.

The non-limiting examples which follow serve to illustrate the invention in more detail and may be read with reference to the figures in which:

FIG. 1 shows the mean values of in vivo release profiles (in beagles) of microparticles each charged with 7-7.5% proinsulin C-peptide and the matrix of which consists of a polymer mixture consisting of PLG polymer RG 503 H+PLG polymer RG 502 H+Mn 2300 in the ratio 80/10/10 (“Commercial Blend”, CB-PLG) or a PLG polymer prepared by ring-opening polymerisation of lactide and glycolide with the addition of 3% dextrane sodium sulphate (DSS) or 4% DSS in the extruder (DSS-PLG).

FIG. 2 shows individual in vivo release profiles (in beagles) of microparticles each charged with 7.5% C-peptide and the matrix of which consists of DSS-modified PLG polymers. FIG. 2a shows four release profiles of 3% DSS-modified polyesters which were produced using 1200 ppm of Sn, FIG. 2b shows release profiles of 3% DSS-modified polyesters produced using 1600 ppm of Sn.

### EXAMPLE 1

### Preparation of Polymers in an Extruder

A. Polymerisation:

First, the DSS is dried for at least 3 days at ambient temperature in a fine vacuum (about 0.1 mbar).

53 mol-% of D,L-lactide, 47 mol-% of glycolide and the predried DSS (2 to 4 wt. %) are weighed into a mixing vessel. To produce a homogeneous mixture the ingredients are mixed for 30 min in a gyrowheel mixer.

The monomer/DSS premix is poured into a metering balance of the extruder (Leistritz double screw extruder type LSM 34 GG) and then metered continuously into the extruder by means of metering screws.

A defined amount of the catalyst tin(II)2-ethylhexanoate is dissolved in toluene and this solution is then continuously metered into the extruder by means of an HPLC pump. The delivery rate is adjusted, taking into account the feed rate, so that the quantity of catalyst in the reaction mixture is 1200 ppm.

The polymerisation is then carried out in the extruder at temperatures between 170 and 220° C.

B. Working up

The crude polymer is dissolved in acetone overnight. After it has dissolved completely, D,L-lactic acid is added and stirring is continued for another 2 to 3 hours. The cloudy solution obtained (10 wt. % of polyester in solvent mixture) is filtered off under a slight nitrogen pressure.

The filtered polyester solution is precipitated with demineralised water. The polyester precipitated is washed, filtered and then dried at a maximum of (35° C.) until a constant weight is achieved.

### EXAMPLE 2

### Preparation of Polymer Mixtures

The following commercially available (Boehringer Ingelheim) polymers were mixed before being formulated by spray drying as described in Example 3 below. In each case, amounts given are in % wt/wt.

(a)=RG 503H: 80

RG 502H: 10

Mn 2300 10

(b) RG 503H 70

RG 502H: 20

Mn 2300: 10

(c) RG 503H: 60

RG 502H: 20

Mn 2300: 20

The release profile for formulation (a) is shown in FIG. 1

### EXAMPLE 3

### Description of the Preparation of Microparticles

0.55 g of polymer, or polymer dry mix, was dissolved in 6 ml of glacial acetic acid, 0.045 g of human C-peptide (C-peptide acetate/Polypeptide Laboratories, Wolfenbüttel, Germany; Cat. No. P-0764) was dissolved in 0.15 ml of water and 3.0 ml of glacial acetic acid and slowly dissolved in the polymer solution. The solution is sprayed at 60° C. in a Buchi 190 spray drier at 60° C. and dried until the microparticles can be obtained as a fine flowing powder.

This method of microparticle production may be used in respect of the polymers produced according to Example 1 or 2.

### EXAMPLE 4

### Delayed-Release Pharmaceutical Formulation

(A) Dry Formulation:

300 mg of microparticles containing 277.5 mg of polymer and 22.5 mg of human proinsulin C-peptide.

(B) Reconstitution of the Dry Formulation to Produce the Delayed-Release Preparation Ready for Use

300 mg of microparticles containing 22.5 mg of human C-peptide are resuspended in 1.5 ml of solvent (0.9% common salt, 0.1% Tween, 0.8% sodium carboxymethylcellulose).

### EXAMPLE 5

### In Vivo Release Rate of C-Peptide from Microparticles

Beagles were subcutaneously given 300 mg of microparticles loaded with 7.5% of human C-peptide. At the specified times, blood was taken from the animals and the C-peptide plasma levels were quantified using LC-MS. The results are shown in FIGS. 1 and 2.

1. A delayed-release pharmaceutical formulation containing proinsulin C-peptide, which is present in an absorbable matrix comprising absorbable polymers wherein said matrix comprises one or more polymers with glycolide and/or lactide units and wherein said matrix is obtainable by mixing lactide and glycolide units in the presence of a tin (II) catalyst and/or an electrolyte containing polyol which acts as a moderator without being incorporated in the polyester or bound thereto in any substantial amount, under conditions which allow polymerisation thereof.

2. A formulation according to claim 1 wherein said polyol which acts as a moderator is dextran sodium sulphate.

3. A delayed-release pharmaceutical formulation containing proinsulin C-peptide, which is present in an absorbable matrix which comprises 2 or more poly lactide glycolide copolymer (PLG) polymers.

4. A formulation according to claim 3 which comprises 3 PLG polymers of different molecular weights.

5. A formulation according to claim 4 which comprises a first polymer with an average molecular weight of between 25 and 35 kDA, a second polymer with an average molecular weight of between 12 and 20 kDA and a third polymer with an average molecular weight of between 1.5 and 3 kDA.

6. A formulation according to claim 5 which comprises 50-80 by weight of said first polymer, 10-30% by weight of said second polymer and 10-20% by weight of said third polymer.

7. A formulation according to one any of the preceding claims, wherein the proinsulin C-peptide is present in a concentration of 1 to 30% (w/w) based on the dry content of the delayed-release formulation.

8. A formulation according to any one of the preceding claims, wherein the delayed-release pharmaceutical formulation comprises microparticles which contain proinsulin C-peptide and an absorbable matrix.

9. A formulation according to claim 8, wherein the microparticles are produced by spray drying.

10. A formulation according to any one of the preceding claims for parenteral administration.

11. A formulation according to any one of the preceding claims for subcutaneous, intracutaneous or intramuscular administration.

12. A formulation according to any one of the preceding claims which provides release, in vivo, of therapeutically relevant amounts of proinsulin C-peptide for at least 5 days.

13. A formulation according to claim 12 which provides release, in vivo, of therapeutically relevant amounts of proinsulin C-peptide for at least 10 days.

14. A formulation according to any one of the preceding claims for treating diabetes or complications of diabetes or for shortening the electrocardiographic QT interval.

15. A formulation according to any one of the preceding claims for treating diabetic neuropathy, diabetic nephthropathy or diabetic retinopathy.

16. A formulation according to one of the preceding claims further comprising another active substance for the prevention, treatment or alleviation of diabetes or complications of diabetes.

17. A formulation, according to any one of the preceding claims, wherein the proinsulin C-peptide is human C-peptide.

18. A formulation, according to any one of the preceding claims, wherein the proinsulin C-peptide is in the form of an acetate salt.

19. A kit comprising a delayed-release pharmaceutical formulation or microparticles according to one of the preceding claims and a device for administering said formulation or microparticles.

20. A method of preparing a pharmaceutical formulation for delayed-release of proinsulin C-peptide which comprises mixing lactide and glycolide units in the presence of a tin (II) catalyst and/or an electrolyte containing polyol, which acts as a moderator, without being incorporated in the polyester or bound thereto in any substantial amount, under conditions which allow polymerisation thereof and combining the polymer mix obtained thereby with proinsulin C-peptide.

21. A method as discussed in claim 20 wherein said polyol is dextran sodium sulphate.

22. A method of preparing a pharmaceutical formulation for delayed-release of proinsulin C-peptide which comprises mixing 2 or more PLG polymers as defined in claims 5 or 6 to form a polymer mix and mixing simultaneously or sequentially combining the polymer mix with proinsulin C-peptide.

23. A method as claimed in any one of claims 20 to 22 wherein the polymer mix and proinsulin C-peptide are mixed by spray drying to form macroparticles.

24. A method as claimed in claim 23 wherein the polymer mix and proinsulin C-peptide are dissolved in solvents and a solvent mixture of both the polymer mix and proinsulin C-peptide is sprayed such that the polymer/peptide mixture is precipitated in particulate form.
